(19) United States
(12) Patent Application Publication
(10) Pub. No.: US 2011/0015754 A1
(43) Pub. Date: Jan. 20, 2011

(54) ARTICULAR IMPLANT COMPRISING AT LEAST TWO CAVITIES

(75) Inventors: Alain LEONARD, Caixon (FR); Freddy Bonnin, Toulouse (FR)

Correspondence Address: BIRCH STEWART KOLASCH & BIRCH, PO BOX 747, FALLS CHURCH, VA, 22040-0747, US

(73) Assignee: TEKNIMED, Vic-en-Bigorre Cedex (FR)

(21) Appl. No.: 12/504,405

(22) Filed: Jul. 16, 2009

(51) Int. Cl. A61F 2/32

(52) U.S. Cl. 623/22.42

(57) ABSTRACT

The present invention relates to a device for the replacement of a permanent articular prosthesis at an implant site, comprising a stem (1) suited to being fixed to a support bone, connected to a head (2) suited to being arranged in an articular area, comprising at least two different cavities (3) with leak tight walls formed at the head (2) made of a thermoplastic material, said cavities (3) emerging at the surface of the device in contact with the implant site, and means for separating (4) cavities (3) from the implant site, enabling the diffusion of a liquid on either side of said separating means.

The present invention relates to articular implants for permanently replacing part of an joint or for maintaining a suitable space for the time necessary for permanent prosthesis replacement operations.

The object of the invention is a ready-to-use implantable device, comprising two assembled modules that provide at least two different active substances, such as two antibiotics.

In patients fitted with an artificial joint, such as for example a hip prosthesis, it is not uncommon to have to replace the implanted element, either on account of its mechanical deterioration, or on account of infectious complications getting into the implant area and leading to the loosening of the prosthesis. It is then necessary to remove the prosthesis and to administer an antiseptic and antibiotic treatment before re-fitting a permanent implant. The whole of this operation, known as “two-stage revision prosthesis”, lasts several weeks or even several months, because it is indispensable before any re-implant to fully treat the infection with a systemic antibiotic therapy combined with a local antibiotic therapy.

The antibiotic treatment may be provided by a new permanent replacement prosthesis or instead a temporary implant, also known as a “spacer”, the purpose of which is to avoid the tissues (muscles, tendons, bone) occupying the space freed by the removal of the permanent prosthesis. In this way the risk of haematoma formation and secondary infection is reduced, and the limb is better stabilized. Furthermore, the patient maintains some mobility during the transitional period and the fitting of the definitive prosthesis is facilitated at a later stage.

Implantable devices are usually made of relatively cheap materials, such as polymer cements, most often polymethylmethacrylate (PMMA in abbreviated form). The use of such a polymer has the advantage of enabling both the maintaining of the implant area and the local treatment of the infection. Indeed, due to its cross-linked structure, it may be combined with an antibiotic agent that diffuses into the surrounding tissues and thus provides a long-term local treatment of the infection. The antibiotic is progressively released by diffusion into the body fluids, which then convey it to neighboring tissues.

The antibiotic systematically used in bone surgery is gentamicin, which is a broad-spectrum antibiotic. However, faced with the increased resistance rates in bacteria isolated in cases of hip infections in particular, recent studies have contemplated the use of a combination of antibiotics in PMMA spacers, such as for example gentamicin and vancomycin (Bertazzoni Minelli E. et al., Journal of Antimicrobial Chemotherapy, 2004, 53, pp. 329-334). For this purpose, commercially available pre-molded spacers loaded with gentamicin have been drilled with holes and these have been filled with a vancomycin-containing cement, this technique allowing to avoid interferences in the release of the two antibiotics by the PMMA and to adjust the elution rate for each antibiotic. These tests have demonstrated the effectiveness of the combined use of two antibiotics in the local treatment of infections at the level of articular prostheses during a two-stage revision, including with respect to strains resistant to conventional treatments. However, the introduction of the second antibiotic into a spacer pre-loaded with gentamicin necessitates delicate and not very reproducible handling, which runs counter to safety and the quality of the treatment.

Several drawbacks emerge during the use of these devices based on antibiotic-loaded polymer cements. The manufacturing process of these PMMA implants indeed requires the mixing of a liquid phase comprising a methacrylate monomer with another solid phase comprising the polymer. This process involves restrictive handling operations, some of which have to be carried out at the moment of the operation so that the surgeon can determine the nature and the quantity of antibiotics that need to be incorporated in the cement. The mixture obtained may exhibit variations in consistency, due to the mixture being formed in a more or less uniform manner, which has consequential effects on the quality of the implant in terms of mechanical strength and diffusion capacity of the loaded active substance.

Once the different components have been mixed together, the cement is poured into molds corresponding to the different parts of the implant. After drying, the part(s) is(are) removed from the mold, inevitably leaving a number of surface irregularities, particularly at the joint lines of the molds used. When there are too many such irregularities, it is necessary to polish the implant prior to its fitting so that the joint may work with the greatest fluidity.

Some polymers are loaded by the health team at the moment of the operation so as to best adapt the antibiotic treatment, which has the aforementioned handling drawbacks, particularly in terms of the production time and the surface quality of the spacer. In other cases, the polymers are ready-made and loaded with active substance selected prior to the operation. The surgeon has available a large set of parts of different sizes and active substances that he or she assembles at the moment of the operation prior to implanting them so as to adapt to the anatomy of the patient and to the type of infection that has to be treated.

In this latter case, the polymers pre-loaded with active substances require specifically controlled storage and transport conditions, especially on account of the fragility of the stored active substances, particularly sensitive to storage temperatures and conditions. The commercialization and the use of these pre-loaded devices are moreover regulated and subject to marketing authorizations, in the same way as any other medicine. The regulatory aspect may represent an obstacle to the use of these products.

It has thus appeared necessary to provide a ready-to-use articular implant, having good surface qualities, not requiring complex handling in the operating theatre, and enabling at least two antibiotics to be provided in local treatment, without substantially increasing the production costs. Such an articular implant must enable both simple and rapid loading in active substances, preferably at the moment of the operation, and enable a good diffusion of the antibiotics in contact with the implant area, without said antibiotics interacting together when they are stored in the implant.

The implantable device of the present invention provides a solution to these requirements by proposing a device composed of two complementary parts liable to be assembled in a single gesture to form a complete implant comprising two different antibiotics loaded in specific areas of the implant, which will enable an efficient local treatment of infections of the articular area.

Not only does the present invention provide a ready-to-use articular implant, providing at least two different active substances, it also has other advantages stemming from the modularity of the device. In particular, it provides the possibility of choosing the two antibiotics to combine at the moment of the operation, thanks to a rapid loading that may be carried out without tedious mixing of cements or resorting to other complicated subsidiary devices. It also makes it possible to have available a range of implants of different sizes, without having to keep a large stock.

Another advantage of the invention is to provide an implant that can be mass-produced, without lengthening or making more intricate the technological process, with a material that is both tough and easy to work, so as to remain within reasonable cost prices. In particular, it is advantageous to avoid technological difficulties in the manufacturing process linked to the homogenization of powder mixtures, the form in which the active substances are generally incorporated in the cement.

This is resolved according to the present invention by the implant being prefabricated with an easily-machinable non-porous thermoplastic material provided as granulates, in which are drilled cavities intended to receive an active substance. The problem of the uniform distribution of the active substances and of the uniformity of mechanical properties and surface appearance of the cements obtained is thus reduced, no mixing between the thermoplastic material and the active substances being carried out before the loading thereof in the spacer cavities provided for such purpose (this latter step being carried out at the moment of the operation). The mechanical characteristics of the implant are fully controlled since they depend directly on the thermoplastic material.

The implants according to the present invention may be marketed and handled without any regulatory authorization being obtained beforehand. Their transport and storage are simplified in so far as they are not subject to restrictive conditions. The loading with active substances is only carried out at the moment of the operation, which allows the surgeon to choose in a more precise manner the type and the quantity of active ingredients that he or she considers necessary to treat the implant area.

Another advantage of the invention is the ability to better control the characteristics of the active substances, these no longer being subject to a random diffusion in a more or less porous matrix, the internal structure of which depends on the quality of the manufacturing process of the cement and particularly the uniformity of the mixing of the powder phase and the liquid phase. Any possibility of interference between the different active substances is eliminated, each of the active substances being compartmentalized in a specific cavity.

Thus, the object of the present invention is a device for the replacement of a permanent articular prosthesis at an implant site, comprising a stem suited to being fixed to a support bone, connected to a head suited to being placed in an articular area, wherein it comprises at least two distinct cavities with leak tight walls formed at the head made of a thermoplastic material, said cavities emerging at the surface of the device in contact with the implant site and means for separating cavities from the implant site, enabling the diffusion of a liquid on either side of said separating means.

The device according to the invention can take any shape suitable to make a permanent or temporary articular implant, whether known at the present time to those skilled in the art or not, this being imposed to a large extent by the anatomy of the joint to be treated. In the present application, “joint” designates the assembly constituted of the neighboring regions of two bones cooperating to ensure a flexible connection of the skeleton. It may be a hinge-type joint such as the knee, where the heads of two bones slide against one another during bending, or instead a spheroidal type joint, also known as a “ball and socket joint”, such as the hip. In this case, the head of femur, or ball, is maintained in the acetabulum (cotyloid cavity) of the pelvic bone and slides inside the socket. The implant according to the invention is intended to replace a bone end, may this end cooperate with another bone end or with a socket. Since the bone repair of the hip is by far the most widespread, it will be specifically described here, although the present invention is explicitly intended for the repair of any joint liable to receive a permanent or temporary implant.

By analogy with anatomical vocabulary, “head” of the implant herein designates the rounded end intended to replace the head of the bone. The stem is the part to be inserted into the support bone and can moreover partly replace the latter to provide the necessary length. The head and the stem form an assembly, namely the implant, their junction being ensured by a simple change of section of the part, with or without change of orientation.

Said at least two cavities are only present on the head of the implant, said head being made of thermoplastic material. This part of the implant is indeed in contact with the implant area the most prone to infection during a two-stage revision prosthesis. Preferably, said at least two cavities are located at an area of the head facing the stem so as to enable the greatest diffusion of the active substances stored in the cavities as possible. Indeed, it is preferred that the separating means are not in direct contact with the friction areas of the joints, so as to facilitate the passage of the active substances from inside the cavities to the implant area. Such a configuration also has the advantage of leaving an articular surface as smooth as possible in contact with the anatomical friction parts of the joint, which favors a fluid movement of the joint.

The preferred thermoplastic material is a polyethylene. This medical grade polymer is initially present in the form of granulates. In this form, and after heating, it can be handled easily and may be worked and machined in large numbers and at reduced cost to shape implants with excellent surface qualities. At one and the same time light, tough and cheap, this material makes a perfect support for an application in the medical field, particularly for prosthetic implants, in complete safety.

Preferably, said at least two cavities have a volume defined by the walls of said cavities and the separating means, ranging between 0.1 and 3 cubic centimeters per cavity. Such a volume allows to introduce into the cavities a sufficient amount of active substance to treat the various infections that may develop during the prosthesis replacement. According to the number of cavities provided on the device, the total volume of active substances stored may be significantly greater than within the scope of a porous polymer cement (up to 30 cubic centimeters within the scope of the invention compared to three times less for a conventional porous cement). Preferably, the volume delimited by the cavities walls and the separating means ranges between 0.3 and 1 cubic centimeters per cavity, which offers the best compromise between the amount of active substance released and the strength of the implant which, under these conditions, is not weakened by the empty spaces constituted by the cavities.

In a specific embodiment of the invention, the separating means are constituted of a cap made of a leak-tight material comprising at least one orifice enabling the diffusion of a liquid. The cap is made of any solid non porous element, resilient or not, capable of preventing the passage of the active substance stored inside the cavity to the exterior, into the implant area. The leak tightness of this element is thus relative, the diffusion of the active substance being obtained by means of at least one orifice present on the cap, the diameter of which varies as a function of the viscosity of the active substance, preferably between 0.1 and 2 mm diameter, and enabling the passage on either side of the separating means. The diameter of the orifice(s) is adjusted according to the active substance stored so as to enable it to diffuse progressively on contact with the body fluids present in the implant medium.

Alternatively, the separating means may be made of a permeable material. In this case, the material involved has a porous structure or is provided with capillaries in order to enable the passage of the active substance from the cavity in which it has been stored to the exterior environment by osmosis or capillarity.

The means for assembling two complementary elements of the implant according to the invention may in theory be located at any level of the implant, for example in the middle of the stem. In a preferred embodiment of the implant according to the invention, the means for assembling two complementary elements are located at the junction of the stem and the head of the implant. This characteristic ensures a number of advantages that will appear more clearly hereinafter, such as the choice of the elements according to their dimension or their loading, ease of preparation and assembly, etc.

The stem and the head of the implant may be connected by simple engagement of one of the two parts in the other. Advantageously, the assembly means are chosen among assembly means suited to being manually joined, with or without the help of a tool. It is thus easy for the surgeon to carry out in a single gesture the fitting of the implant from two elements, instantaneously. The assembly means may be for example constituted of a Morse taper, the male cone being preferably borne by the stem and the female cone by the head. When the assembly means are joined by jamming one element in the other, the necessary force may be provided by an auxiliary tool such as an impactor. The assembly is carried out preferably by simple manual pressure.

In this latter embodiment, according to another advantageous characteristic of the implant according to the invention, the head has a symmetry of revolution in relation to the axis of the junction. Indeed, in this case the relative position of the parts during the assembly may be any position. The relative orientation of the two elements is determined during the molding of the parts by the shape of the junction. Thus, the practitioner can fit the implant very quickly and with complete peace of mind as regards the result of the assembly.

As already mentioned, the shape and the dimensions of the implants are to a large extent imposed by the anatomy of the joint to be treated. Since the standing height of patients is variable, it is necessary for the implant according to the invention to be able to embrace several sizes, and in particular that the dimensions of the head and the stem can vary independently of each other to be associated in all desired combinations. Thus, in particular when the implant is intended to be fitted in the hip, the stem may have a substantially cylindrical or conical shape with a length ranging between 80 mm and 200 mm. Furthermore, the head can fit into a sphere of diameter ranging between 40 mm and 70 mm.

According to a preferred embodiment of the implantable device according to the invention, the stem is made of polymethylmethacrylate. It may comprise in its core a metal reinforcement, preferably made of stainless steel. This reinforcement makes it possible to absorb the forces applied on the implant and to stiffen the assembly.

The structure of the implant such as it has been described has in itself numerous advantages, particularly as regards the possibility of combining elements of different sizes, adapted to the specific anatomy of each patient. The device according to the invention thus comprises at least two different active substances inside said at least two cavities. The active substances are thus combined by incorporation in the different cavities provided for this purpose, without any risk of interference between them after loading, the walls of the cavities being made of the leak tight thermoplastic material.

Preferably, said at least two active substances are chosen among the group of antibiotics, antiseptics, anti-inflammatories, analgesics, antimitotics, anaesthetics or a mixture thereof. Each of these active substances is incorporated in a specific cavity, independently of each other, so that no interference can occur between them inside the implant. This may be carried out at the moment of the operation by means of a pipette before sealing the cavities by the separating means provided for this purpose, or instead by means of a syringe, the needle of which can pass through one of the orifices present on the separating means that will have been fitted beforehand to the empty cavities or instead through the porous membrane of the cap which will be pierced by the needle of the syringe.

The structure of the device according to the invention thus makes it possible to combine as many active substances as there are cavities. This is achieved in an extremely simple manner, without it being necessary to cast as many cements as active substances or instead to arrange numerous prefabricated parts corresponding to the different active substances. The possibilities of combination of active substances are thus much greater than in the prior art and are not liable to weakening the implant assembly or increasing in an ill-advised manner the preparation time of the implant. Obviously, this makes it possible to improve significantly the therapeutic treatment associated with the prosthesis replacement, particularly by cumulating the effects of the different active substances used that could play a complementary role with differential diffusion times.

Thanks to the present invention, a hospital establishment can thus have available a range of heads and stems comprising different antibiotics or other active substances, of different sizes and that can be combined according to requirements. This modularity allows the health team to choose, depending on the infection, the specific antibiotic, in the required amount, without it being necessary to have available a large stock. A surgical kit for the replacement of a permanent articular prosthesis comprising a plurality of devices according to the invention of different dimension and/or shapes may be used advantageously in this respect. This allows to implement a more efficient treatment for the patient and to give more freedom of action to the health team, while providing them with a material of high quality, very uniform, devoid of mechanical weaknesses and ensuring a regular diffusion of active ingredients.

The following examples illustrate the invention so as to better understand its characteristics and advantages, without limiting its scope in any way.

The following figures also help to illustrate the present invention without limiting its scope:

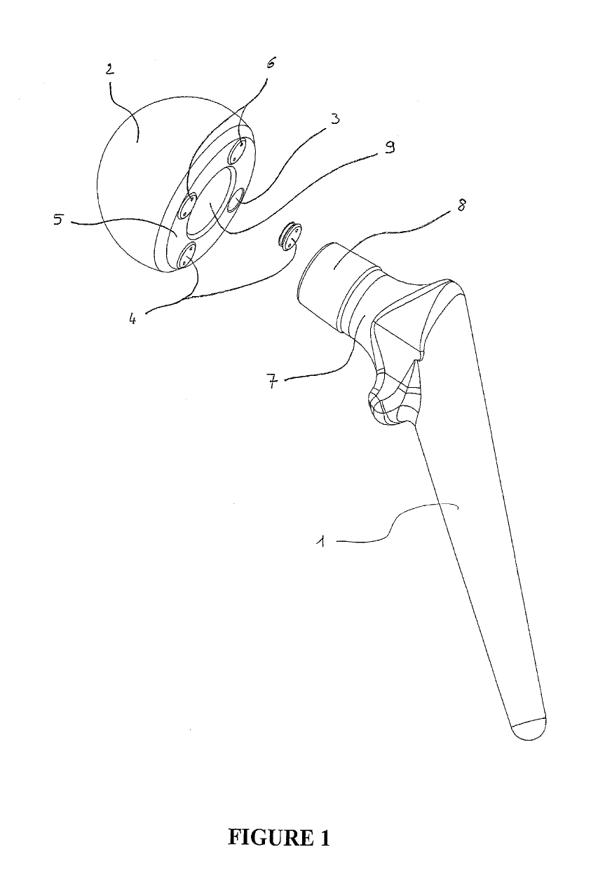

FIG. 1 illustrates a hip implant according to the invention, before assembly.

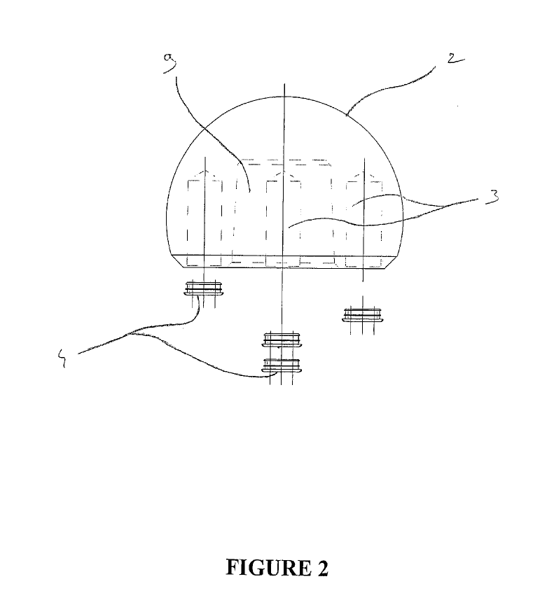

FIG. 2 illustrates the head of a hip implant according to the invention, with the separating means not assembled, in side view.

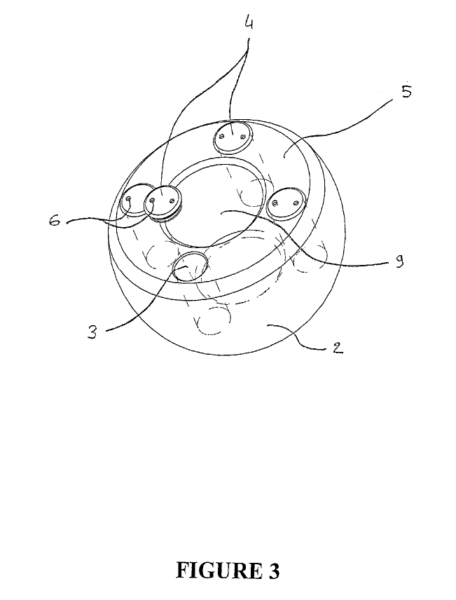

FIG. 3 illustrates the head of a hip implant according to the invention, with the separating means assembled, in bottom view.

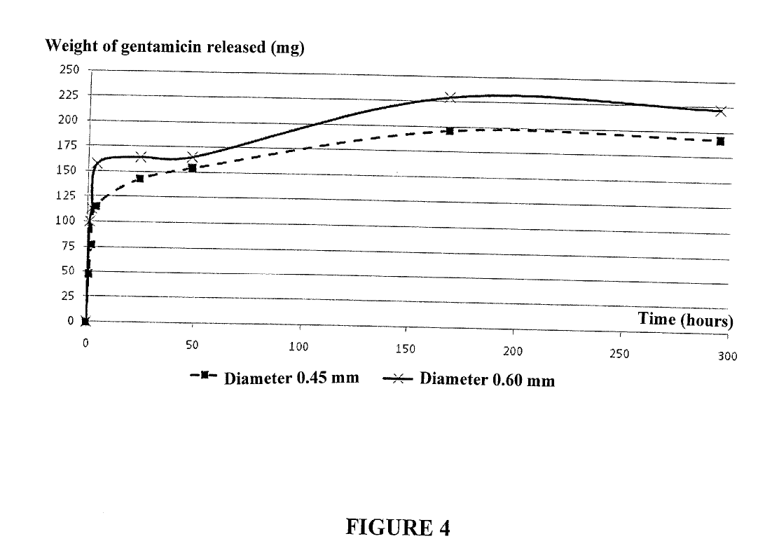

FIG. 4 illustrates the release kinetic of an antibiotic incorporated in an implant according to the invention over a period of 12 days.

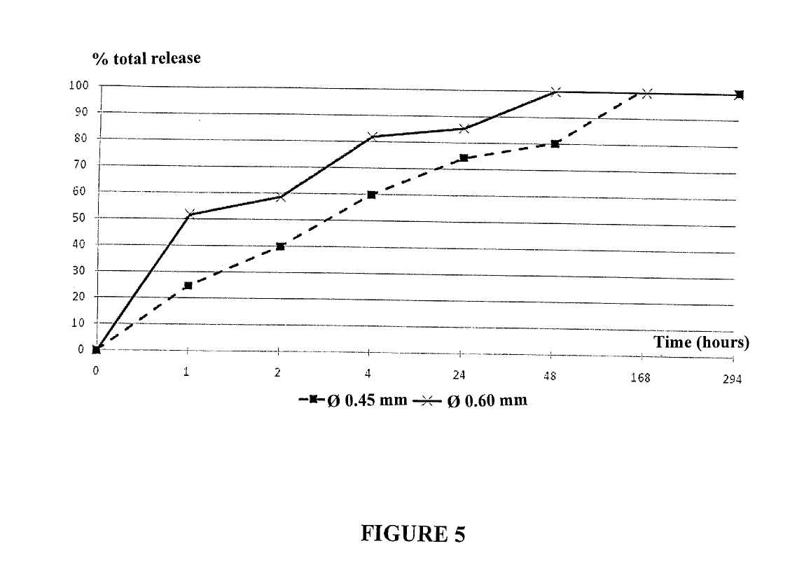

FIG. 5 illustrates the percentage release corresponding to the kinetic of FIG. 4.

### EXAMPLE 1

### Structure of a Hip Implant According to the Invention

The implantable device for the replacement of a hip prosthesis as illustrated in FIGS. 1, 2, and 3, comprises:
a stem 1 made of PMMA suited to being secured to the support bone, here the femur, not comprising any cavity, and
a head 2 made of polyethylene suited to being positioned in the articular area, provided with four cavities.

These two elements are complementary and comprise the means for rigidly assembling together arranged at the junction 7 of the stem 1 and the head 2 of the implant. The assembly means are constituted of a Morse taper, the male cone 8 being borne by the stem 1 and the female cone 9 by the head 2, which enables the assembly of the implant by simple manual pressure, or with the help of an impactor.

The stem 1 has a substantially conical shape and ends at its proximal extremity with the junction 7 comprising the male cone 8. It has a length of 120 mm and a diameter of 20 mm. It comprises in its core a stainless steel metal reinforcement (not represented) of a section varying from 6 mm to 10 mm. The head 2 has a symmetry of revolution in relation to the axis of the Morse taper. It fits into a sphere of 54 mm diameter (2), or alternatively of 60 mm (2′) or 48 mm (2″) diameter. It has four cavities of 1 cubic centimeter each liable to be loaded with four different active substances. Each of the cavities is sealed by a polyethylene cap provided with two orifices of 0.6 mm diameter.

### EXAMPLE 2

### Diffusion Test of an Implant According to the Invention

The implant described in example 1, comprising a polyethylene head of 48 mm diameter, is loaded with a total volume of 8 mL of 80 mg/mL gentamicin spread out in the four cavities sealed by polyethylene caps provided with orifices of 0.45 mm diameter.

Another implant identical to the first is loaded with the same quantities of gentamicin but with cavities sealed by polyethylene caps provided with orifices of 0.60 mm diameter.

The diffusion of gentamicin from each of these two implants is measured in liquid medium (physiological saline solution) over a period of 12 days. The results are illustrated in FIGS. 4 and 5.

As of the first hours, a significant release of gentamicin is observed, which may be described as a “flash” effect. The larger the diameter of the orifices, the more important is the effect. The diffusion proceeds in a regular manner over time with high quantities of antibiotic released into the liquid medium. These quantities remain higher with the implant sealed by caps provided with orifices of 0.6 mm diameter. They remain higher than those of the implant sealed by caps provided with orifices of 0.45 mm diameter up to the end of the diffusion test.

1. A device for the replacement of a permanent articular prosthesis at an implant site, comprising a stem suited to being fixed to a support bone, connected to a head suited to being arranged in an articular area, wherein said device comprises at least two distinct cavities with leak tight walls formed at the head made of a thermoplastic material, said cavities emerging at the surface of the device in contact with the implant site, and means for separating cavities from the implant site, enabling the diffusion of a liquid on either side of said separating means.

2. A device according to claim 1, wherein said at least two cavities are located at an area of the head facing the stem.

3. A device according to claim 1, wherein the thermoplastic material is a polyethylene.

4. A device according to claim 1, wherein said at least two cavities have a volume delimited by the walls of said cavities and the separating means ranging between 0.1 and 3 cubic centimeters per cavity.

5. A device according to claim 1, wherein the volume ranges between 0.3 and 1 cubic centimeters per cavity.

6. A device according to claim 1, wherein the separating means are constituted of a cap made of a leak tight material comprising at least one orifice enabling the diffusion of a liquid.

7. A device according to claim 1, wherein the separating means are constituted of a cap made of a permeable material.

8. A device according to claim 1, wherein the stem is made of polymethylmethacrylate.

9. A device according to claim 1, wherein the stem has a metal reinforcement in its core.

10. A device according to claim 1, wherein the metal reinforcement is made of stainless steel.

11. A device according to claim 1, wherein the stem is connected to the head by engagement.

12. A device according to claim 1, wherein the stem is connected to the head by assembly means suited to being manually joined, with or without the help of a tool.

13. A device according to claim 1, comprising at least two different active substances inside said at least two cavities.

14. A device according to claim 1, wherein said at least two active substances are chosen among the group of antibiotics, antiseptics, anti-inflammatories, analgesics, antimitotics, anaesthetics or a mixture thereof.

15. A surgical kit for the replacement of a permanent articular prosthesis, wherein said kit comprises a plurality of devices according to claim 1, of different dimension and/or shapes.

16. A device according to claim 2, wherein the thermoplastic material is a polyethylene.

17. A device according to claim 2, wherein said at least two cavities have a volume delimited by the walls of said cavities and the separating means ranging between 0.1 and 3 cubic centimeters per cavity.

18. A device according to claim 3, wherein said at least two cavities have a volume delimited by the walls of said cavities and the separating means ranging between 0.1 and 3 cubic centimeters per cavity.

19. A device according to claim 2, wherein the volume ranges between 0.3 and 1 cubic centimeters per cavity.

20. A device according to claim 3, wherein the volume ranges between 0.3 and 1 cubic centimeters per cavity.
